(19) United States
(12) Patent Application Publication
(10) Pub. No.: US 2009/0095309 A1
(43) Pub. Date: Apr. 16, 2009

(54) Therapeutic/diagnostic external airway position support (EAPS) device and method

(76) Inventors: Steven J. Derrick, West Mifflin, PA (US); Joyce A. Derrick, West Mifflin, PA (US)

Correspondence Address: Gary P. Topolsky, 4031 Brownsville Road, Pittsburgh, PA, 15227, US

(21) Appl. No.: 12/286,710

(22) Filed: Oct. 1, 2008

Related U.S. Application Data
(60) Provisional application No. 60/999,089, filed on Oct. 16, 2007.

(51) Int. Cl. A61F 5/37

(52) U.S. Cl. 128/870

(57) ABSTRACT

A hands free, external airway position support device for use on a patient undergoing a surgical, therapeutic and/or diagnostic procedure. The device adheres to the patient at the base of his or her chin, jaw or neck so that no wrap around neck or head sling attachment means are required. When installed, the device will extend the patient's neck and maneuver his or her chin upward and outward, away from the base of the neck. As this device will be relatively inexpensive to manufacture and not overly complicated, it can be disposed of after use rather than requiring sterilization for reuse.

### CROSS-REFERENCE TO RELATED APPLICATIONS

This application claims priority to U.S. Provisional Application Ser. No. 60/999,089, filed on Oct. 16, 2007, the disclosure of which is fully incorporated by reference herein.

### BACKGROUND OF THE INVENTION

1. Field of the Invention

This invention relates to anesthesia delivery and a device for adjustably supporting the mandibular region of a patient to minimize airway compromises during surgical therapeutic and/or diagnostic procedures. It may also be utilized to assist a practitioner via position support/stabilization during mask ventilation or laryngoscopy. The invention further relates to an improved method for using the aforesaid device during and after certain surgeries.

2. Background of the Invention

Monitored Anesthesia Care (or “MAC” anesthesia) and Total Intravenous Anesthesia (or “TIVA”) are two common means for delivering appropriately titrated doses of drugs to a patient along with anesthetic topicalization or infiltration by the surgeon at the operative site. MAC anesthesia can be used with localized or regional anesthetics, such as spinals, epidurals, topicals and peripheral nerve blocks, for providing the patient with a temporary numbing or loss of feeling and movement at a preferred, surgical location. One risk of using the MAC or TIVA anesthetic technique is that an upper airway compromise can, and often does, occur. And an unattended interference with a free exchange of respiration predisposes patients to a higher incidence of morbidity/mortality.

During many surgical procedures, upper airway obstruction can and does occur, in varying degrees, with patients whose level of consciousness is depressed by sedation/MAC/TIVA techniques. Upper airway obstruction is usually caused by a loss of tone in both the external and internal neck (or sub-mandibular) musculature, i.e., those muscles that provide direct and indirect support to the skull base itself, the mandible, the tongue and soft palate, as well as the structures of the pharynx and larynx. Collectively these structures are referred to as the Anterior Triangle of the Neck (ATN). Because of this diminished tonicity, the upright and balanced position of the jaw and skull are negatively impacted upon while the tongue and soft palate begin to approximate the posterior pharyngeal wall thereby compromising free respiratory exchange. Hyoid cartilage musculature also relaxes resulting in the epiglottis obstructing the larynx. One basic technique known for keeping the patient's airway open is to tilt the head back, lift the chin upwards and anteriorly displace the mandible. This is sometimes referred to as the triple airway maneuver.

For keeping the patient's airway unobstructed, many anesthetists start with the aforementioned chin-lift maneuver, i.e., manually lifting and then holding the patient's chin upwards. That maneuver maintains proper head tilt and anterior mandible displacement resulting in improved alignment of the airway structures. Another option, the jaw-thrust maneuver, is performed by an anesthetist placing his or her hands at both sides of the patient's mandible and laterally thrusting the jaw forward. Both methods require the anesthetist to manually provide external airway position support to varying degrees throughout the duration of the surgery.

In some MAC/TIVA anesthesia cases, an oropharyngeal or nasopharyngeal airway may be employed to maintain and improve airway patency. An oropharyngeal airway is a plastic, disposable, semi-circular shaped device that, when properly situated, will hold the tongue away from the posterior wall of the pharynx. The nasopharyngeal airway remedies similar obstructions occurring in the region of the soft palate. Even with the use of such devices, the patient's head, neck and chin must still be maintained in a proper alignment. Surgical procedures that employ MAC/TIVA techniques can range from a few minutes to several hours in duration. During that period, the anesthetist must continuously administer sedative medications, assess the patient's response to same as well as changes in the level of surgical stimuli, all the while monitoring and documenting vital signs on the patient's chart. Other factors, such as the surgical logistics, can make performing the noted airway maneuvers awkward and preclude the anesthetist from more efficiently performing his/her other responsibilities.

Still other disadvantages posed by the necessity to manually prevent airway obstruction include: restricting ergonomics of the anesthetist by requiring near constant contact with the patient's head, neck and mandibular region, thereby impeding the anesthetist from performing his or her other tasks, such as delivering medications and observation and monitoring of patient response as well as procedural documentation. The patient's relative positioning on the operating room table can make it even more difficult to maintain constant upward deflection of the chin especially during prolonged surgical procedures. The anesthetist's arms, hands and fingers can become unnecessarily fatigued and/or stiff from having to maintain the required application of forces for airway maintenance for prolonged intervals.

For the foregoing needs, it is evident that the patient's mandibular support should create an upward extension (albeit with only the slightest of force) to cause his or her head to remain tilted backwards during the surgical procedure. That is why the semi-flexible but not adjustable, hands-free chin lift of Reddick U.S. Pat. No. 6,969,366 is wedged under the patient's chin and against the upper chest, or manubrial region. The foregoing device was an alternative to the pitchfork-like chin support banded behind the patient's neck in Rotramel U.S. Pat. No. 6,171,314, and the steel framed, neck support of Davies U.S. Pat. No. 4,782,824. And all of those represent a significant leap in patient comfort over the banded, sling support means depicted in Carden U.S. Pat. No. 5,632,283.

In other instances, neck supports have been designed and patented for non-anesthetic reasons. For instance, the one piece cervical appliance of Mundell U.S. Pat. No. 4,700,697 is meant to assist the wearer overcome sleep apnea concerns. A soft but solid molded support block can be strapped about the wearer's neck for non-medical reasons according to Williams U.S. Pat. No. 7,055,908. It represents an improvement over the chin resting sling of Palley U.S. Pat. No. 4,565,408. And the ventilated neck brace of Bugarin U.S. Pat. No. 6,409,694 claims adjustability along its width and length.

Numerous other cervical collars include the one piece foam rubber neck wrap of Cheatham U.S. Pat. No. 6,926,686, a predecessor model per Newton U.S. Pat. No. 4,232,663, and a spring-loaded, wrap around collar like that shown in Gorsen U.S. Pat. No. 4,827,915. There is also the wrap around, split assembly of Bender U.S. Pat. No. 5,275,581. The cervical collar of Martin U.S. Pat. No. 6,726,643 claims automatic adjustability; while in Garth et al. U.S. Pat. No. 7,141,031, that cervical collar has an end-supported chin strap.

The inflatable cervical device of Rogachevsky U.S. Pat. No. 5,752,927 has four separate chambers for holding the wearer's neck in a neutral position, i.e. substantially horizontal or forward facing, during traction. It has no obvious contact at its support base with the wearer's upper chest/manubrium and its chin support is an “add on” that would not appear to provide any neck/mandible support regardless of the degree of inflation imparted therein. Finally, this prior art device would not be easy to use. Unlike the present invention, the prior art device would require fastening, with Velcro straps, completely about the neck of its wearer. With such complicated “plumbing”, it would be expected to require sterilization before reuse, rather than being disposable per se.

### SUMMARY OF THE INVENTION

It is a principle object of this invention to provide an external airway position support device that will overcome the shortcomings of the prior art devices. It is another object to provide that device with some versatility, in terms of relative height extendibility. Another principle object is to provide a device that will allow a practitioner to monitor and control the juxtaposition of the head, chin and neck during procedures in which lesser or greater degrees of sedation/analgesia are required.

Still another principle object is to provide an external airway position support that can be held in place, beneath the patient's mandibular region, with adhesive means rather than requiring any around-the-neck straps of any sort and/or over the top of the head slinging mechanisms like those shown in the prior art.

Another object of this invention is to provide an external airway position support device that is easy to use on a patient while sitting, partially reclining, supine or laying lateral to one degree or another, either face up or face down. Another object is to provide an external airway position support that is lightweight and inexpensive to make, use and operate. A still further object is to provide a position support device that is readily disposable after use.

With the various embodiments of this invention described below, an anesthetist can more efficiently provide patient care during MAC and/or TIVA anesthesia, as this device makes it unnecessary for the anesthetist to continuously perform the required intricate airway maneuvers. Accordingly, several main objects and advantages of the present invention include: (a) not requiring the anesthetist to maintain substantially continuous physical contact with the patient's external airway at all times; (b) allowing the anesthetist less restricted movement around the patient's positioning; (c) freeing up the anesthetist to perform his or her other tasks, including administering medications, procedural observation, monitoring and documentation of respective details and responses; (d) rendering less critical the choreographing of operating room table placement and patient positioning in order to maintain the appropriate force vectors on the external airway during long surgical procedures; (e) preventing the anesthetist's arms, hands and fingers from becoming unnecessarily fatigued or stiff as would otherwise be the case when having to continually provide manual airway support; (f) avoiding the need to induce general anesthesia when local anesthesia would otherwise suffice with supplemental sedation/analgesia thereby alleviating other potential detriments such as sore throat, an increased propensity to nausea/vomiting, and injury to dentition or mucosal structures; (g) providing stable, balanced and constant external airway position support in all patients who undergo MAC/TIVA techniques; and (h) providing a disposable method for hands-free support of the external airway allowing proper alignment of anatomic structures of the airway, resulting in improved respiratory exchange, thereby preventing and/or resolving obstruction.

Further objects and advantages are to provide a device which can be used easily and safely, which is inexpensive to manufacture, and because of same, is readily disposable after use. The device may also be used to provide airway positioning support during mask ventilation and for assisted positioning during laryngoscopy. Still further objects and advantages will become apparent from a consideration of the ensuing description and drawings.

The present invention is a hands free, external airway position support device for use on patients undergoing surgical, therapeutic and/or diagnostic procedures. It is meant to adhere to the patient at the base of the jaw and at the base of the neck so that no wrap around neck or head sling attachment means are required. Preferred embodiments are one or two piece foam units held in place with glue or adhesive between the patient's chin/neck or jaw and upper chest/mandible. More preferred embodiments employ sections of hook and loop (or Velcro) tape with the latter sections being glued directly onto the patient's chin and upper chest. Alternate embodiments may include a plurality of inflatable segments that alone, or in combination with additional foam components, assist in extending the patient's neck and maneuvering his/her chin upward and outward. As this device will be relatively inexpensive to manufacture and not overly complicated in design and/or mechanics, it can be disposed of after use rather than requiring sterilization for reuse.

### BRIEF DESCRIPTION OF THE DRAWINGS

Further features, objectives and advantages of the present invention will become clearer when referring to the following detailed description of preferred embodiments made with reference to the accompanying drawings in which:

FIG. 1 is a perspective side view of a first embodiment of unitized EAP support device according to the invention properly installed between a patient's chin and manubrial regions;

FIG. 2A is a schematic side view of the unitized manubrium arch and crescent lever component from FIG. 1;

FIG. 2B is a schematic side view of the two piece manubrium arch and crescent lever components from a second embodiment of EAP support device according to this invention;

FIG. 3 is a top perspective view of the manubrium arch component from FIG. 2B;

FIG. 4 is a bottom perspective view of the manubrium arch component from FIGS. 2B and 3;

FIG. 5 is a front schematic view of the FIG. 3 manubrium arch component positioned on a patient's upper chest or manubrium;

FIG. 6 is a top perspective, right side view of the crescent lever component from FIG. 2B;

FIG. 7 is a side schematic view of the crescent lever component from FIG. 6 flexed downwardly prior to installation between a patient's chin and upper chest;

FIG. 8 is a top schematic view of an embodiment of chin/mandible grip component for the present invention;

FIG. 9 is a front schematic view of the chin/mandible grip component from FIG. 8 as it would appear when adhesively bonded to a wearer's chin according to the invention;

FIG. 10 is a close up, left side schematic view of the chin/mandible grip component from FIG. 9 held in place beneath the wearer's chin;

FIG. 11 is a front perspective view of a third embodiment of mandibular lift device according to this invention;

FIGS. 12A, B and C are side schematic views of the mandibular lift device from FIG. 11 positioned between a wearer's chin and upper chest prior to inflation (FIG. 12A); at a midpoint of inflation (FIG. 12B); and at or near full inflation (FIG. 12C);

FIG. 13 is a top perspective view of a fourth embodiment of mandibular lift device according to this invention;

FIG. 14 is side schematic view of the FIG. 13 device positioned about the neck of a patient; and

FIG. 15 is a flowchart depicting the installation method steps for one embodiment of this invention.

### DETAILED DESCRIPTION OF PREFERRED EMBODIMENTS

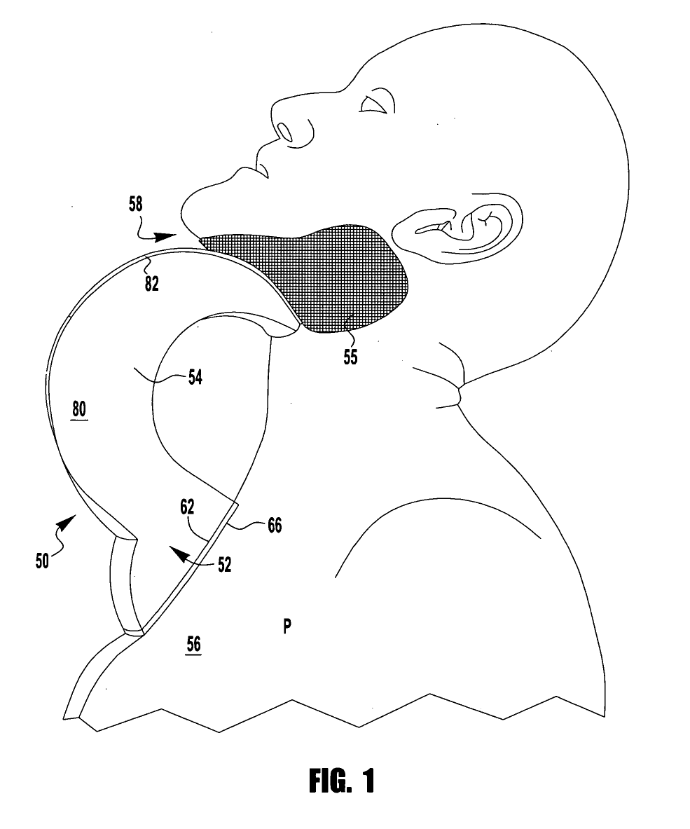

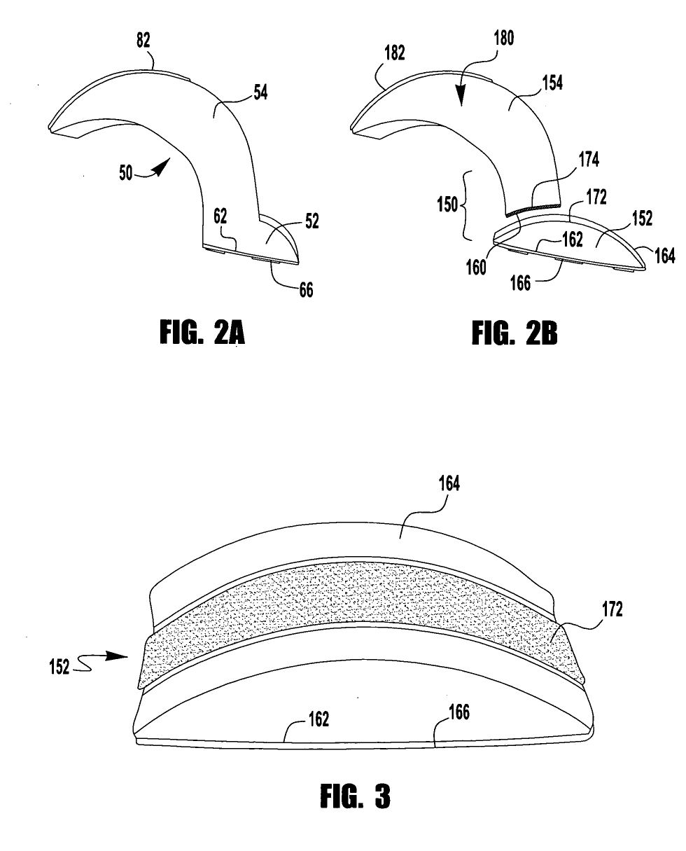

Referring to FIGS. 1 and 2A, there is shown a first preferred embodiment of external airway position support device, generally 50. In this version, the two main components, a manubrium arch 52 and crescent lever 54, are unitized, i.e. made from one section of material such as foam or the like. This one-piece device 50 serves as an EAP support when situated at the base (or underside) of crescent lever 54 to the wearer/patient's manubrium 56 and beneath said wearer/patient's chin, jaw or mandible region 58. Device 50 may be glued directly to the wearer/patient P at one or both ends. Such adhesion provides for a strong connection between manubrium 56 and mandible 58 regions. Alternately, one or both ends of device 50 may include sections of hook and eye tape (or Velcro) with corresponding sections of tape glued to the manubrium 56 and/or mandible 58 regions of patient P as explained herein. A representative section of such tape 55 is glued to the patient's chin or mandible 58 in FIG. 1. It would adhere to a corresponding section of Velcro tape 82 at the uppermost curve for the crescent lever arm 80 of crescent lever 54.

Another section of Velcro tape 66 adheres to the bottom 62 of manubrium section 52 as shown. The underside to manubrium arch 52 is kept substantially planar for better resting on, and adhesion to, the patient's manubrium 56 for many therapeutic/diagnostic surgical procedures. Optionally, device 50 may be left in place for continued airway support of the patient P in the post-anesthesia care unit. In alternate embodiments (not shown), the manubrium section of this device may adhere directly to a rectangular adhesive patch glued to the patient's upper chest. At least one of the manubrium base and the upper surface of said patch should be provided with one or more known surgical adhesives. In some instances, an adhesive-to-adhesive connection may be desired, or even preferred, over the Velcro-to-Velcro manubrium connection described above.

The main elements for manubrium arch 52 and flexible crescent lever 54 can be fabricated from a flexible, elastically expandable foam-like material. Representative examples of same include, but are not limited to: a plastic, such as polyethylene, polyvinyl chloride, rubber and/or similar natural and synthetic substances. As flexibility is less critical for the manubrium arch 52, it can be made from still other alternative materials suitable for temporarily adhering to a base of flexible crescent lever 54.

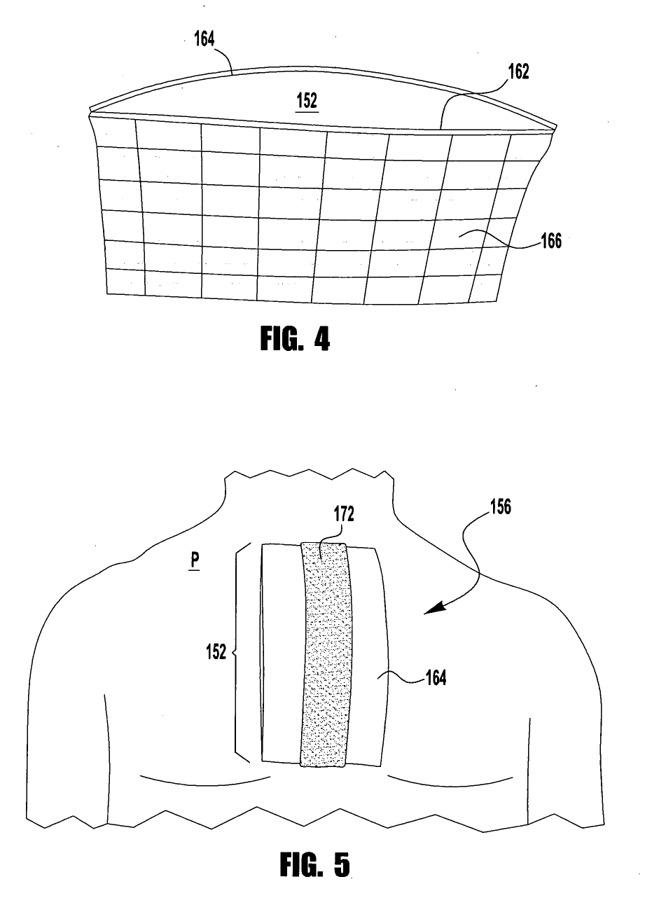

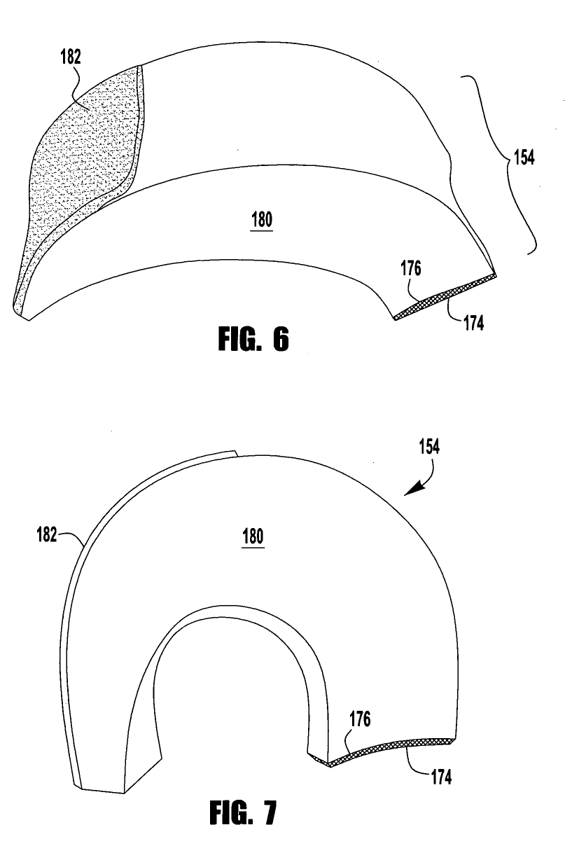

Referring to FIGS. 2B through 7, there is shown a second embodiment of the present invention. In the FIGURES for alternate embodiments, common components are commonly numbered, though in the next “hundred” series. As such, the second version of this invention for an EAP support device, generally 150, has two separate and distinct elements, a manubrium arch component 152 (as best seen in accompanying FIGS. 3-5) and a crescent lever component 154 that are joined together with corresponding sections of Velcro tape, generally 160, adhered to adjoining ends of said components 152 and 154, as best seen in FIGS. 2B, 3 and 5. With a flexible crescent lever component 154, this two-piece device 150 can also serve as a suitable EAP support when situated at its base to manubrium arch component 152 and then to a chin/mandible grip 155 beneath a wearer/patient's mandible region 158, the latter grip component being similar to the one used with the first embodiment in FIGS. 1 and 2A.

As shown in FIGS. 3 through 5, this embodiment of manubrium arch 152 has a flat underside 162 and curved upper surface 164. Alternatively, upper surface 164 of manubrium arch 152 may consist of several consecutive planar portions giving that upper surface more of a polygonal appearance (not shown). To the flat underside 162, there is included a first adhesive layer 166 of such composition that any existing gaps/contours may be filled. First adhesive layer 166 enables the device 150 to be fixedly, albeit temporarily, installed to the manubrium 156 of patient P, slightly below the anterior base of that patient's neck.

To the upper surface 164 (whether curved or polygonal), there is positioned a wide section 172 of hook and loop (or Velcro) tape. Tape section 172 joins to a corresponding Velcro component 174 on the lowermost base 176 of flexible crescent lever 154. That lowermost base can be made substantially planar. Alternatively, it can be provided with a slight concave, or inward arch for better adhering to the section of Velcro tape positioned on the arched or curved upper surface 164 of manubrium arch 152.

Per FIGS. 6 and 7, a curved, slightly flexible crescent lever arm section 180 extends upwardly from the lowermost base 176 of crescent lever 154. As the chins/upper neck regions of patients vary in size, width, etc., it is possible to make crescent lever components in varying widths and/or lengths. This is especially true when using this device for younger, smaller wearer/patients.

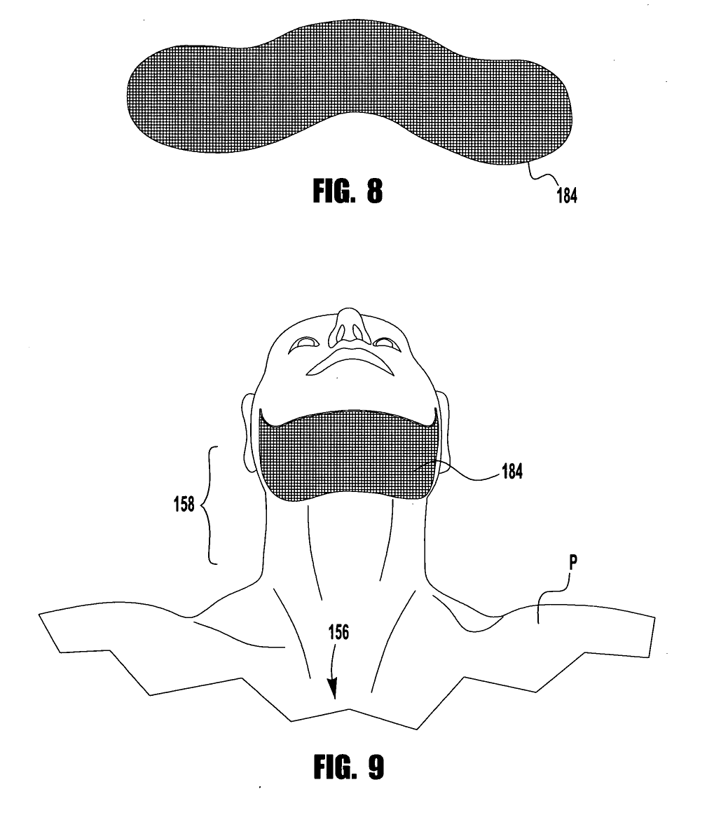

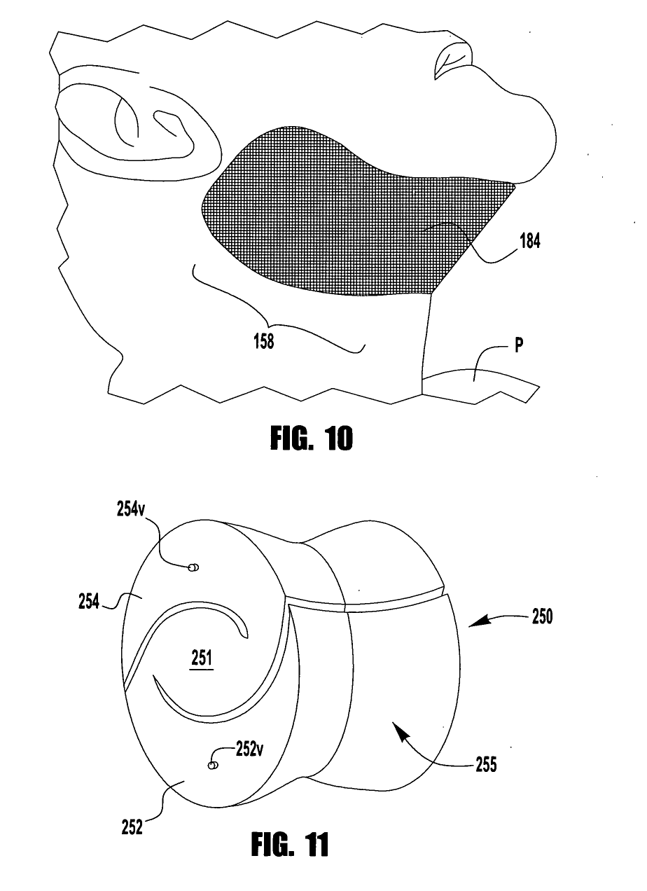

Atop an uppermost region of crescent lever arm section 180, particularly on the outside of same, there is positioned a strip of Velcro tape 182. It is meant to attach to the corresponding central Velcro component 184 on the outside and underside of chin/mandible grip 155 adhesively secured to the wearer/patient's chin and/or uppermost neck 188. See especially, FIGS. 8 through 10. In the embodiment shown, nearly half of the specially curved configuration for chin/mandible grip 155 is covered with a Velcro tape, hook and loop material, from side edge to side edge. Alternatively, only the center most regions of chin/mandible grip 155 need to have a matching Velcro component 184.

Several surgically acceptable adhesives are known. Many alternatives allow the anesthetist and surgical team to select from a menu of options in the event a patient may know of past allergic reactions to some. The use of adhesives is preferred over past known attachment methods for many cervical collars. These past collars were belted about the wearer's neck, and connected with Velcro® straps, buckles and/or snaps. They would preclude a rapid removal of the device in the event of a surgical emergency. Ideally, it is preferred that the patient/wearer's neck be kept accessible for just such an emergency. In still other known devices, one or more elastic bands were used to “sling” the wearer's chin upward.

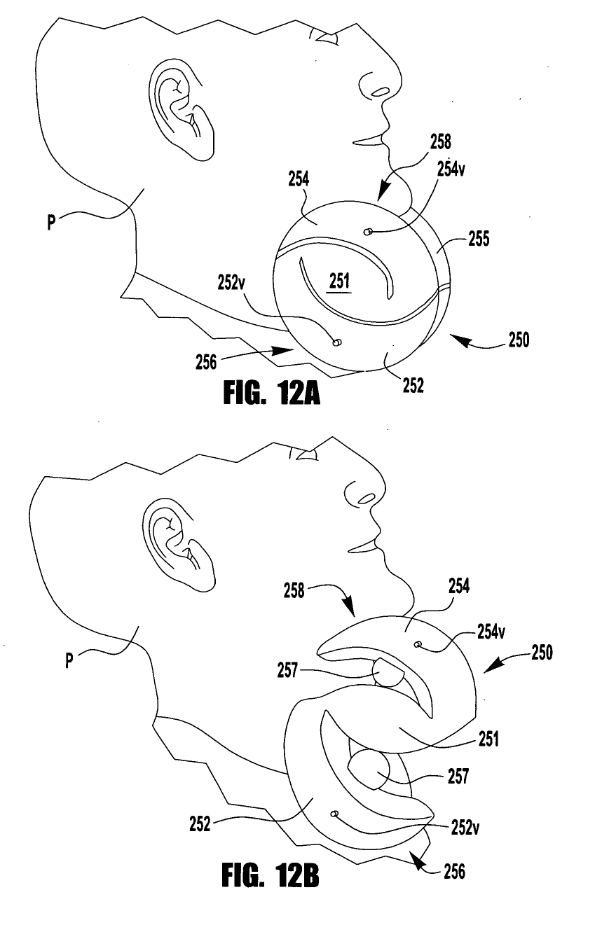

In accompanying FIGS. 11, 12A, 12B and 12C, there is shown a third embodiment of EAP device, generally 250, that has an overall circular appearance in top view. Before inflation, device 250 has a somewhat Z-shaped design with a fattened or rounded, central region 251. It may also resemble the symbol for a hurricane on weather maps.

Device 250 may be made from an inflatable plastic liner preconfigured to have a lowermost chest contacting, or manubrium portion 252 and an upper or top mandibular portion 254 on opposite sides of central region 251. The side edge 255, between front and rear faces to device 250, is contoured to resemble a continuous V-shape. With that configuration, device 250 can serve as a suitable chin-rest when positioned beneath the mandibular region 258 of patient P.

As shown, both the lower portion 252 and top portion 254 have separate valves, 252v and 254v, respectively. When connected to a pump, gas can or other air supplying means (not shown), each valve causes a ball shaped bladder 257 to expand thereby forcing either arm portion, the lower chest contacting 252 or top mandibular portion portion 254, to further separate or duly spread apart from the main central region 251.

This alternate design for an EAP support device is relatively reversible. With its universal V-shaped side edge 255, the entire device 250 can be rotated 180 degrees for situating its lower chest contacting portion 252 against the patient's manubrium 256, and its upper mandibular portion 254 against the patient's mandible 258. In yet another alternate (not shown), reversibility is not as critical. Therein, the device side edge would not be uniformly V-shaped, but only cupped inwardly for serving as a patient chin rest. The side edge to the lowermost portion would be kept substantially planar for better resting, and adhesion, to the patient's manubrium.

It is understood that the valves for the foregoing embodiment may be situated on the body proper of device 250 for inflation with a bulb inflation mechanism and/or a large, luer-lock type syringe as is available in most operating rooms. Also, depending on the valves and maximum pressures desired, a mere can of compressed air could rapidly couple and fill device 250 after it is duly situated on at least one side to the patient's chest or chin. For greater control, it is preferred that the same valve through which air is added can serve as a “bleeder valve”, through which only some air can be removed if slightly overfilled, or through which most of the air can be extracted after the surgery has finished. The device might also be left in place for continued airway support in the post-anesthesia care unit.

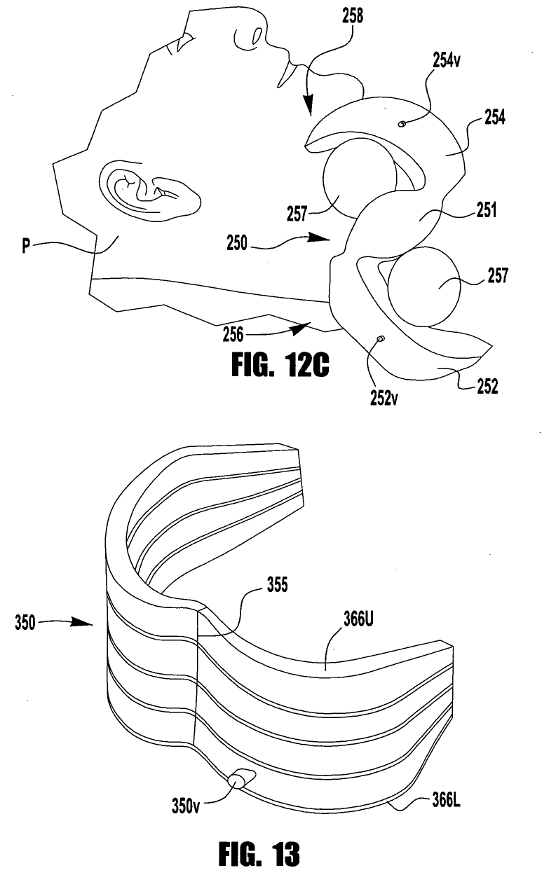

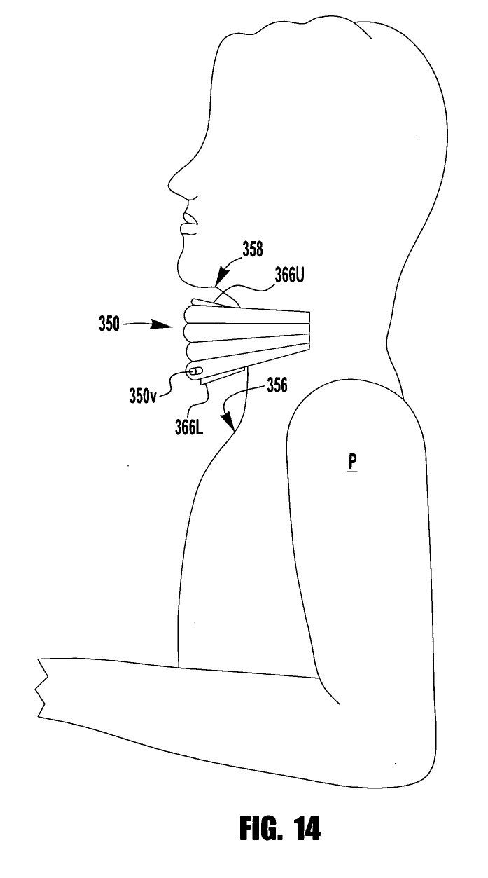

Referring to FIGS. 13 and 14, there is shown a fourth embodiment wherein device 350 has a plurality of chambers 350c that upon inflation will accordion upwardly from a starting position to expand between the patient's upper chest 356 and chin 358. As shown, device 350 is essentially C-shaped, though in the embodiment at FIG. 13, a structural vertical reinforcement 355 gives the device 350 somewhat of a hinged appearance. Regardless, it is preferred that regardless of the number of baffles comprising device 350, they should all be filled (and subsequently deflated) through a single, common valve 350v.

For illustrative purposes, the lower end of device 350 is provided with a first layer of adhesive 366L for sticking to a patient's manubrium 356 and/or partially about the anterior base of their neck. On the opposite end, the uppermost surface of device 350 has its own adhesive layer 366U for adjoining same to the wearer's chin, or mandibular region 358.

Any of the foregoing devices 50, 150, 250 or 350 can be fabricated from a flexible, elastically expandable material. Representative examples of same include, but are not limited to: a plastic, such as polyethylene, polyvinyl chloride, rubber and/or similar natural and synthetic substances.

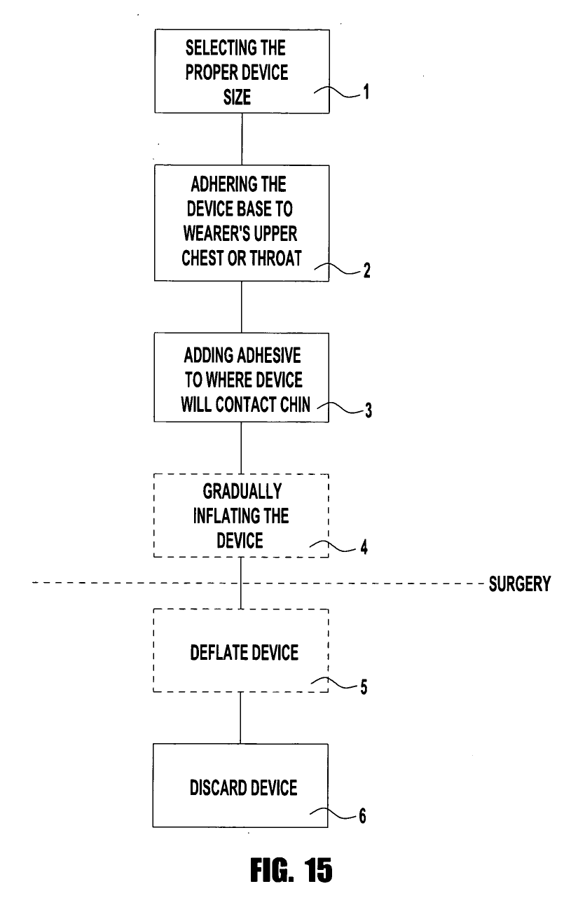

FIG. 15 is a block diagram chart showing one preferred method for installing the device of this invention on a wearer/patient. It includes the steps of:
1. Selecting the proper size of device for the patient's neck dimensions (i.e. adult versus child, thinner versus heavier wearer, etc.).
2. The device is adhered at its base to the patient's upper chest or throat.
3. Adhesive is next applied to where the upper regions of this device will contact with the underside to the patient/wearer's chin or mandible.

For the inflatable embodiments at FIGS. 11 through 14, next step 4 calls for gradually inflating the device such that through expansion, the patient's neck is progressively extended and his/her mandible advanced upwardly and outwardly to the required degree. As this step is unnecessary for the non-inflatable device versions shown at FIGS. 1 through 7, it is duly marked with dashed lines in the process flowchart steps of FIG. 15.

Following completion of the surgery, optional step 5 for the inflatable versions calls for purposefully deflating the device (either fully or partially). Finally, step 6 entails discarding a fully removed device rather than subjecting it to sterilization and reuse.

In some instances, it may prove prudent to leave any of these devices on the patient proper through some portion of post-anesthesia care and post-surgical recovery. In that manner, the device and method of this invention can further assist in providing needed, supplemental support for a recovering patient's airway passages.

The present invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A device for assisting in maintaining optimal external airway position (EAP) support during surgical, therapeutic and/or diagnostic procedures, said device adhering, directly or indirectly, to a patient's upper chest at one end and to an underside of the patient's chin at another end for keeping the patient's chin a preferred distance apart from the patient's upper chest when the device is installed therebetween.

2. The device of claim 1 which comprises a manubrium attaching component and a mandible attaching component.

3. The device of claim 2, wherein the manubrium attaching component and the mandible attaching component are integrally formed.

4. The device of claim 2, wherein the mandible attaching component fixedly attaches to a section of the manubrium attaching component for installation of the device between the patient's chin and upper chest.

5. The device of claim 2, wherein the mandible attaching component is structurally bendable.

6. The device of claim 2, wherein at least one of said manubrium attaching component and said mandible attaching component has an expandable element.

7. The device of claim 6, wherein said expandable element includes one or more inflatable chambers.

8. The device of claim 6 which further includes a V-shaped chin recess.

9. The device of claim 2, wherein the manubrium attaching component includes an arch for the patient's upper chest.

10. The device of claim 2, wherein the mandible attaching component includes a crescent-shaped lever.

11. The device of claim 2, wherein said manubrium attaching component and said mandible attaching component are made from a material selected from the group consisting of: a polyethylene, polypropylene, polyvinyl chloride, nylon, rubber, a laminated fiber, cardboard, paper and combinations thereof.

12. The device of claim 2, wherein at least one of said manubrium attaching component and said mandible attaching component adhere to sections of hook and loop material that are directly adhered to the patient.

13. A device for assisting in maintaining optimal external airway position (EAP) support during surgical, therapeutic and/or diagnostic procedures, said device comprising:
a manubrium attaching component for adhering, directly or indirectly, to a patient's upper chest at one end; and
a mandible attaching component for adhering, directly or indirectly, to the patient's chin at another end for keeping the patient's chin a preferred distance apart from the patient's upper chest when the device is installed therebetween.

14. The device of claim 13, wherein the manubrium attaching component and the mandible attaching component are integrally formed.

15. The device of claim 13, wherein the mandible attaching component fixedly attaches to a section of the manubrium attaching component for installation of the device between the patient's chin and upper chest.

16. The device of claim 13, wherein the manubrium attaching component includes an arch for the patient's upper chest; and the mandible attaching component includes a crescent-shaped lever.

17. The device of claim 13, wherein said manubrium attaching component and said mandible attaching component are made from a material selected from the group consisting of: a polyethylene, polypropylene, polyvinyl chloride, nylon, rubber, a laminated fiber, cardboard, paper and combinations thereof.

18. The device of claim 13, wherein at least one of said manubrium attaching component and said mandible attaching component adhere to sections of hook and loop material that are directly adhered to the patient.

19. A method for assisting in maintaining optimal external airway position support for a patient during surgical, therapeutic and/or diagnostic procedures, said method comprising the steps of:
(a) selecting an appropriately sized device for the patient's neck and chest dimensions, said device comprising: (i) a manubrium attaching component for adhering, directly or indirectly, to the patient's upper chest at one end; and (ii) a mandible attaching component for adhering, directly or indirectly, to the patient's chin at another end for keeping the patient's chin a preferred distance from the patient's upper chest once the device is fully installed;
(b) adhering the device at one end to the patient's upper chest; and
(c) adhering the device at the other end to the patient's chin.

20. The method of claim 19, wherein the order of adhering steps (b) and (c) are reversed.
